(19) United States
(12) Patent Application Publication
(10) Pub. No.: US 2014/0242656 A1
(43) Pub. Date: Aug. 28, 2014

(54) METHOD FOR PRODUCING ALCOHOLS AND/OR SOLVENTS FROM PAPER PULPS WITH RECYCLING OF THE NON-HYDROLYSATED PLANT MATERIAL IN A REGENERATION REACTOR

(71) Applicants: Marcel ROPARS, Palaiseau (FR); Caroline AYMARD, Lyon (FR); Anais GUILLAUME, Rueil-Malmaison (FR); Sandra MENIR, Gonesse (FR)

(72) Inventors: Marcel ROPARS, Palaiseau (FR); Caroline AYMARD, Lyon (FR); Anais GUILLAUME, Rueil-Malmaison (FR); Sandra MENIR, Gonesse (FR)

(73) Assignee: IFP ENERGIES NOUVELLES, Rueil-Malmaison Cedex (FR)

(21) Appl. No.: 14/269,450

(22) Filed: May 5, 2014

Related U.S. Application Data
(63) Continuation of application No. 13/518,703, filed on Sep. 28, 2012.

(30) Foreign Application Priority Data
Dec. 23, 2009 (FR) 09/06.334

(51) Int. Cl. C12P 7/14; C12P 7/16; C12P 7/28

(52) U.S. Cl. CPC C12P 7/14; C12P 7/28; C12P 7/16
USPC 435/150; 435/162

(57) ABSTRACT

This invention describes a process for the production of alcohols and/or solvents from cellulosic or lignocellulosic biomass that comprises at least the following stages:
a) Alkaline chemical pretreatment of a cellulosic or lignocellulosic substrate;
b) Optionally washing of the pretreated substrate;
c) Enzymatic hydrolysis of the substrate that is pretreated and optionally washed using cellulolytic and/or hemicellulolytic enzymes that produce a hydrolyzate and a water-insoluble residue;
d) Microorganism fermentation of the hydrolyzate that is obtained from stage c) and production of a fermentation must that contains at least one alcohol and/or solvent;
e) Separation/purification of alcohol and/or solvent, and
f) Separation of a cake that contains the insoluble residue,
   in which at least a portion of the cake that is obtained in stage f) is sent into at least one reactor for regeneration of cellulose, before being recycled downstream from stage a) for alkaline chemical pretreatment.

### FIELD OF THE INVENTION

This invention is part of the framework of a process for the production of so-called “second generation” alcohol and/or solvent from lignocellulosic biomass. It relates more particularly to a process for the production of ethanol and/or an acetone-butanol-ethanol mixture (also called an ABE mixture).

### PRIOR ART

The lignocellulosic biomass represents one of the most abundant renewable resources on earth. The substrates that are under consideration are very varied, since they relate both to ligneous substrates (leafy and resinous), the by-products of agriculture (straw), or those of the lignocellulosic waste-generating industries (farm produce and papermaking industries).

The lignocellulosic biomass consists of three primary polymers: cellulose (35 to 50%), hemicellulose (20 to 30%), which is a polysaccharide that consists essentially of pentoses and hexoses, and lignin (15 to 25%), which is a polymer of complex structure and high molecular weight, consisting of aromatic alcohols that are connected by ether bonds.

These different molecules are responsible for intrinsic properties of the vegetation wall and are organized in a complex intergrowth.

Cellulose and optionally hemicelluloses are the targets of enzymatic hydrolysis but are not directly accessible to the enzymes. This is the reason for which these substrates are to undergo a pretreatment preceding the enzymatic hydrolysis stage. The purpose of the pretreatment is to modify the physical and physico-chemical properties of the lignocellulosic material for the purpose of improving the accessibility of the cellulose that is imprisoned within the matrix of lignin and hemicellulose.

Numerous technologies for carrying out this pretreatment exist: acid baking, alkaline baking, vapor explosion, organosolv methods, etc. The effectiveness of pretreatment is measured both by the material balance at the end of the pretreatment (recovery level of sugars in soluble monomer or oligomer form or in insoluble polymer form) and also by the cellulosic and hemicellulosic residues' susceptibility to enzymatic hydrolysis.

The processes for the production of alcohols and/or solvents from lignocellulosic biomass, called “second-generation processes,” comprise at least the following stages:
Pretreatment of the substrate,
Enzymatic hydrolysis of the pretreated substrate,
Fermentation of the hydrolyzate that is obtained, and
Separation/purification of the alcohol and/or solvent that is obtained after fermentation.

The economic validity of this type of process for the production of alcohol and/or solvent is difficult to achieve even for the operators that have broad mobilizable resources. Two items have a strong impact on overall expense: the enzymatic feedstock that is necessary for the hydrolysis of polymerized sugars and the pretreated plant material. The optimization of this type of process therefore requires optimum upgrading of the enzymatic feedstock that is expressed in terms of kg of sugars released per kg or FPu of added enzymes. These conditions are produced by means of low enzymatic feedstocks, typically 5 to 10 g/kg of dry material. Unfortunately, these slow enzymatic feedstocks do not enhance the pretreated substrate well because the hydrolysis yield is mediocre, in particular that of the glucans that constitute the essential target because the conversion of glucose into ethanol and ABE is easy.

The insoluble dry material that is subjected to enzymatic hydrolysis can vary by 5 to 40%, and in general between 10 and 25%. According to the publication by Kristensen et al. Biotechnology for Biofuels, 2009 (2) 11, for obtaining an identical hydrolysis yield, the enzymatic consumption is to be higher in the case of an elevated feedstock of insoluble dry material, in particular because of the deactivation of the enzyme by the products of enzymatic hydrolysis (glucose, cellobiose). The approach that would consist in carrying out dilution at the enzymatic hydrolysis stage is, however, limited since it will have significant consequences on the energy cost linked to the separation of the alcohol that is produced by distillation. In the specific case of the manufacturing of ethanol, an alcohol concentration of the fermentation must with 23-25 g/L of ethanol at a minimum (alcohol titre of 3) is necessary to ensure a reasonable cost for the distillation item.

In contrast, to optimize these processes, it is desirable to maximize the amount of hydrolyzable and enzyme-accessible raw material from an initial amount of biomass.

Beyond the improvements linked to the effectiveness of enzymatic cocktails, the improvement of the economic balance sheet of the production of ethanol or ABE can be obtained by means of recycling of different flows or products.

The Patent Application WO 94/29475 proposes an improved process for the conversion of cellulosic biomass into ethanol in which a portion of the effluents that are obtained from the fermenter is recycled at the inlet of the same fermenter as a source of nutrients for the microorganism that is used during the fermentation.

In the second-generation processes, to improve their economic profitability, an effort is made to maximize the amount of released sugars, and therefore to maximize the yield, while keeping consumption of products and enzymes the lowest possible.

This invention describes an improved process for the production of alcohols and/or solvents that is paired with a unit for the production of papermaking pastes, using an alkaline chemical pretreatment.

### SUMMARY OF THE INVENTION

This invention relates to a process for the production of so-called second-generation alcohols and/or solvents, in which the lignocellulosic or cellulosic biomass undergoes an alkaline pretreatment, and then a recycling of the pastes that are not hydrolyzed enzymatically is performed, after said pastes pass into a reactor for regeneration of cellulose.

### DESCRIPTION OF THE DRAWINGS

FIG. 1 is a diagrammatic representation of a device that implements a process for the production of alcohols and/or solvents from papermaking pulps, comprising a stage for recycling the solid residues, according to this invention.

### DETAILED DESCRIPTION OF THE INVENTION

This invention describes a process for the production of alcohols and/or solvents from cellulosic or lignocellulosic biomass that comprises at least the following stages:
a) Alkaline chemical pretreatment of a cellulosic or lignocellulosic substrate;
b) Optionally washing of the pretreated substrate;
c) Enzymatic hydrolysis of the substrate that is pretreated and optionally washed using cellulolytic and/or hemicellulolytic enzymes that produce a hydrolyzate and a water-insoluble residue;
d) Microorganism fermentation of the hydrolyzate that is obtained from stage c) and production of a fermentation must that contains at least one alcohol and/or solvent;
e) Separation/purification of alcohol and/or solvent, and
f) Separation of a cake that contains the insoluble residue,
   in which at least a portion of the cake that is obtained in stage f) is sent into at least one reactor for regeneration of cellulose, before being recycled downstream from stage a) for alkaline chemical pretreatment.

Thus, owing to the process according to this invention, it is possible to improve the upgrading of the lignocellulosic substrate. Actually, the process makes it possible to use more than 80% by weight, and preferably more than 90% by weight, of the cellulose that is contained in the vegetation for its future alcohol conversion, and/or in an ABE mixture.

Owing to the thermal treatment carried out in the alkaline medium, under mild conditions, in a specific so-called cellulose-regeneration reactor, the cellulose that is not hydrolyzed, also called recalcitrant cellulose, partially recovers its susceptibility to enzymatic hydrolysis. The term of recalcitrant cellulose, in the meaning of this invention, is defined as cellulose that is not hydrolyzed during stage c) for enzymatic hydrolysis and that has, without specific treatment, a mediocre susceptibility to enzymatic hydrolysis.

According to the process of this invention, a fraction of the cake that is extracted in stage f) is sent into at least one reactor for regeneration of cellulose before being recycled downstream from stage a) for alkaline pretreatment: the non-hydrolyzed recalcitrant cellulose thus undergoes a heat treatment, in an alkaline environment, under milder conditions than those used during the pretreatment stage. This treatment makes possible in particular the swelling of the fibers of the paste and regenerates the susceptibility of the substrate to enzymatic hydrolysis, without giving rise to the accumulation of lignin.

The fraction of the cake that is extracted in stage f) that is sent into the regeneration reactor is mixed with an alkaline solution and then heated to a temperature of between 50 and 150° C., preferably for a period that varies between 10 minutes and 4 hours.

The alkaline solution is preferably sodium sulfate. The baking temperature in the regeneration reactor is then between 70 and 150° C., preferably for a dwell time in the reactor of between 0.5 and 4 hours.

The alkaline solution can also be gaseous ammonia. In this case, the temperature is between 50 and 100° C., preferably for a dwell time of between 10 and 60 minutes.

The alkaline solution can also be a percolated ammonia solution, heated to a temperature of between 80 and 140° C. in the regeneration reactor.

The process according to this invention makes it possible to limit the amount of enzymes that are to be used for producing an overall hydrolysis of greater than 90% of the cellulose of the initial pretreated substrate. Ultimately, the enzyme only encounters a substrate that is “very susceptible to enzymatic hydrolysis” and no longer encounters recalcitrant cellulose because of the recycling of the latter.

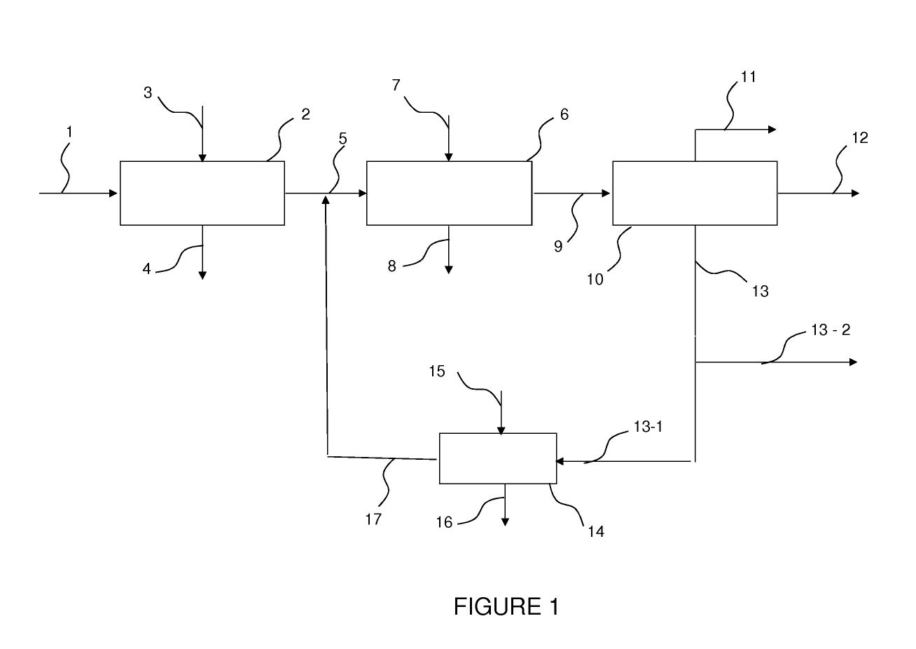

The invention will be described by referring to FIG. 1.

The substrate that is used is selected from among the most varied biomasses, but more particularly from the resinous arborescent types (softwood such as spruce or pine) or leafy arborescent types (hardwood such as eucalyptus) or else agricultural lignocellulosic waste (corn straw, rice, etc.).

The pretreatment that is carried out in stage a) is an alkaline-type pretreatment. This first stage is a stage for baking cellulosic or lignocellulosic substrate in the presence of an alkaline chemical reagent. This reagent comes in liquid or gaseous form, based on the pretreatment that is used.

According to one embodiment, the alkaline chemical pretreatment that is carried out in stage a) is preferably a pretreatment with sodium sulfate (or Kraft process) that is conventionally used in the processes for production of papermaking products, called Kraft or “sulfate paste,” at the end of which papermaking pastes are obtained.

The sodium sulfate process or Kraft process is based on the use of soda and sodium sulfate. The chemical treatment of wood chips is done at 150-180° C. for a period of 1 to 7 hours based on the substrate that is used. The Kraft papermaking pastes are produced from the most varied biomasses but more particularly from the resinous arborescent types (softwood such as spruce or pine) or leafy arborescent types (hardwood such as eucalyptus) or else agricultural lignocellulosic waste (corn straw, rice, etc.). They are partially delignified by means of baking at high temperature and in the presence of soda. This delignification is monitored by the operating parameters of the reactors. The baking is carried out in a vertical reactor, where the chips drop by gravity and encounter the various baking liquors. The sodium sulfide is prepared directly from sodium sulfate by combustion. During baking, the sodium sulfide is hydrolyzed into soda, NaHS, and H₂S. The different sulfur-containing compounds that are present react with lignin to provide thiolignins that are more easily soluble. The liquor that is applied to the chips is called white liquor. The liquor that is extracted from the reactor or digester that contains the compounds that are eliminated from the wall is called black liquor.

The biomass is introduced via a pipe 1 into the cooking plant or digester 2. The white liquor is also introduced there via the pipe 3. The biomass is partially delignified by means of baking at high temperature and in the presence of soda. The solubilized lignin is removed with the alkaline solution and is discharged via the pipe 4 with the black liquor.

This delignification stage can take place in several successive digesters that are not shown in the figure and is controlled by the operating parameters set in these devices.

The paste that is obtained at the outlets of the digesters circulating in the pipe 5 is enriched with cellulose: it contains between 60 and 90% by weight of cellulose relative to the total solid material and between 5 and 20% hemicellulose.

According to other embodiments, the alkaline chemical pretreatment that is carried out in stage a) can also be a pretreatment by explosion of the fibers with ammonia, also called AFEX (Ammonia Fiber Explosion) pretreatment or a percolation pretreatment that uses ammonia with recycling, also called ARP (Ammonia Recycle Percolation) pretreatment.

The ARP (Ammonia Recycle Percolation) process is a pretreatment process that uses ammonia with recycling. This type of process is described in particular by Kim et al., 2003, Biores. Technol. 90 (2003), pp. 39-47. The high temperature of the percolation leads to a partial solubilization both of lignin and hemicelluloses; this solution is next heated for recycling the ammonia and recovering, on the one hand, the extracted lignin, for example for an energy upgrading, and, on the other hand, the soluble sugars that are obtained from hemicelluloses.

According to this embodiment, in the case of an ARP pretreatment, the biomass is introduced via the pipe 1 into the cooking plant 2. An ammoniacal solution is percolated on the biomass that is pressurized (15 to 30 bar) and at a high temperature, 130° C. to 190° C. The biomass is partially delignified; a portion of the hemicelluloses is also solubilized. The solubilized sugars and lignin are removed with the spent alkaline solution and are discharged via the pipe 4.

The AFEX (Ammonia Fiber Explosion) process consists in introducing the lignocellulosic substrate into a high-pressure cooker in the presence of ammonia, and then causing an explosive pressure relief at the outlet of the reactor, and in recycling the ammonia that is then in gaseous form. This type of process is described in particular by Teymouri et al., 2005, Biores. Technol. 96 (2005), pp. 2014-2018. This process primarily leads to a destructuring of the matrix of the biomass, but there is no phase separation of lignin, hemicellulose and cellulose compounds at the treatment outlet.

According to this other embodiment, in which the pretreatment is of the AFEX type, the biomass is introduced via the pipe 1 into a cooking plant 2. An ammoniacal solution is introduced via the pipe 3 under pressure, from 15 to 30 bar, at moderate temperature (70° C. to 110° C.). The mixture is kept under these conditions for a time period that is determined based on the substrate, and then pressure is released from the mixture at the outlet of the cooker. The ammoniacal solution is recovered via the pipe 4 in gaseous form to be recycled. The pretreated substrate that is extracted via the pipe 5 of the cooker essentially has the same composition as the substrate at the input.

The pretreated substrate according to an AFEX or ARP process that circulates in the pipe 9 comprises between 50 and 95% by weight of water-insoluble materials, and more particularly between 60 and 85% by weight of water-insoluble materials.

Other alkaline treatments are also under study, in particular based on soda or chalk; a non-exhaustive review is provided by Ogier et al., 1999, Oil & Gas Science and Technology—Review of the FPI, Vol. 54 (1999), No. 1, pp. 67-94.

These different alkaline pretreatments can be combined with a mechanical action, created, for example, in a two-screw-type extruder or a defibering unit.

At the end of the pretreatment stage a) according to the process of this invention, the pretreated substrate is obtained in the form of a cellulose-enriched paste 5 (also called “pulp”).

During the washing stage b) of the pretreated substrate, this paste that circulates in the pipe 5 is optionally washed in the reactor 6. One or more washing liquids 7 are introduced into said reactor 6. This washing stage can also be repeated several times, optionally in several successive washing reactors. It can also be limited to a dilution stage.

A more intensive delignification can be conducted during the washing stage that is carried out in the reactor 6. A separation tool such as a press or a centrifugal decanter can be installed for eliminating the alkalinity.

The washing stage b) is necessary if the alkaline pretreatment is a Kraft- or ARP-type pretreatment.

The spent washing liquid(s) 8 is/are removed at the outlet of the reactor 6.

In the case of a Kraft-type pretreatment, the paste or washed pulp 9 that is extracted from the washing reactor 6 contains between 1% and 40% of solid material, preferably between 7% and 40%, and more preferably between 10% and 25%.

In the case of an AFEX treatment, the washing stage b) after this pretreatment can be limited to a dilution stage with a dilution liquid that is introduced via the pipe 7, in which case the flow of washing liquid discharged via the pipe 8 is zero.

A neutralization of the paste can be conducted prior to the stage for enzymatic hydrolysis by the addition of acids. It is actually necessary that the enzymatic hydrolysis be carried out at a pH of between 4 and 5.5.

The pretreated and optionally washed and neutralized paste is next sent into the process for conversion of alcohols and/or solvents, shown diagrammatically by the rectangle 10, where the stages c) to e) are carried out, corresponding to the conversion stages themselves. These conversion stages can be two to eight in number. Preferably, there are between three and five of them.

These conversion stages comprise at least the stages c) and d) that correspond respectively to an enzymatic hydrolysis and a fermentation of the pulp. These stages can optionally be coupled in the same reactor. Reference is then made to the SSF (“Simultaneous Saccharification and Fermentation”) process.

The enzymatic hydrolysis stage c) is carried out by means of enzymes of the cellulases and/or hemicellulases type produced by a microorganism. In a preferred way, the microorganism that is used is a mushroom that belongs to the genera Trichoderma, Aspergillus, Penicillium or Schizophyllum, or an anaerobic bacteria that belongs to the genus Clostridium. In a very preferred way, the microorganism that is used is Trichoderma reesei. It is produced in an independent production line that can be set up onsite or offsite.

Using the alkaline pretreatment, the susceptibility to enzymatic hydrolysis is excellent, and the cellulose and hemicellulose polymers are converted into sugars called “very fermentable” (glucose, mannose), “poorly fermentable” (galactose), and “hardly fermentable” (xylose and arabinose). The enzymatic hydrolysis conditions, primarily the level of dry material of the mixture to be hydrolyzed and the amount of enzymes used, are selected in such a way that stage c) is carried out so that a conversion of between 20% and 90% of the cellulose of the pulp that circulates in the glucose pipe 9, and more particularly between 30% and 80%, is obtained.

The alcohol fermentation carried out in stage d) is ensured by yeasts or other microorganisms.

During stage e), the alcohols and/or solvents that are produced in stage d) are purified and separated.

Stage f) for separation of the cake can be carried out downstream from stages c), d) and/or e) and can optionally be coupled to a washing of the cake.

In all of the cases, at the outlet of stages c) to e) that are carried out in the reactor 10, a flow of products 11, optionally separated by any means that is known to one skilled in the art, a liquid residue 12 (called vinasse) containing unfermented sugars, and a solid cake 13 containing the solid material that is obtained from the initial substrate (solid residue), and a liquid fraction are obtained. The solid residue partly consists of cellulose that has not been hydrolyzed and that represents between 10% and 100% of the solid, and preferably between 30% and 70%.

The flow 13 that corresponds to the cake is divided into 2 fractions 13-1 and 13-2.

The fraction 13-1 is sent to the reactor 14 for regeneration of the cellulose. This fraction represents between 20 and 100% of the cake 13, and preferably between 75 and 100%, and even more preferably between 85 and 100%.

An alkaline solution is introduced into the reactor 14 via the pipe 15, to be mixed with the fraction 13-1.

The flow that exits from this reactor is recycled via the pipe 17 downstream from stage a) for alkaline chemical pretreatment.

Thus, owing to the treatment in the regeneration reactor that makes possible in particular the swelling of the fibers, the recalcitrant cellulose that is contained in the flow 13-1 partially recovers its susceptibility to the enzymatic hydrolysis.

The non-recycled fraction 13-1 is directly discharged beyond the process. It represents between 0 and 80% of the cake 13, and preferably less than 25%, and, even better, less than 15%.

### EXAMPLES

In all of the examples below, dry material is denoted as ms.

FPu=Filter Paper Unit, which is a measurement of the enzymatic activity. The FPu-weight correspondence is a characteristic of the enzymatic cocktail.

### Example 1

### Material Balance—without Recycling (Not in Accordance with the Invention)

A process for the production of ethanol from papermaking pulp obtained from a Kraft alkaline process is considered. The process treats 80 tons/hour of native vegetation. The vegetation is spruce (softwood), containing 55% by weight of dry material that consists of:

| | |
|---|---|
| Cellulose | 42% |
| Lignin | 30% |
| Hemicellulose | 15% |
| Others (Ashes, | 13% |
| Extractibles) | |

The hemicelluloses consist of 50% mannans.

The Kraft baking is carried out at 175° C. for 5 hours. This pretreatment and the washing processes carried out in stages a) and b) respectively are conducted in such a way that the papermaking pulp contains 15% dry material, and has preserved the following:

| | |
|---|---|
| Cellulose | 97% |
| Lignin | 10% |
| Hemicellulose | 52% |
| Others | 8% |

The ethanol conversion process consists of enzymatic hydrolysis of the papermaking pulp (stage c)) followed by alcohol fermentation into ethanol (stage d)), a separation of the solids in suspension for forming a cake, distillation, and then dehydration of ethanol at 99.7% by weight (stage e)).

The enzymatic hydrolysis is conducted under conditions such that the hydrolysis of 75% of cellulose and 55% of hemicelluloses is observed. 20 FPu/g of cellulose that enters into the hydrolysis reactor is consumed.

The fermentation makes it possible to transform 90% of the previously formed glucose and mannose into ethanol. The other sugars that are obtained from the hemicelluloses (xylose, arabinose, . . . ) are not fermented by the Saccharomyces cerevisiae strain that is used.

Before the distillation stage, the solid residue is separated and washed to limit the loss of ethanol with the cake.

The conditions of the process are such that the exiting flows are:
Ethanol at 99.7% by weight: 6.96 tons/hour
Vinasse: 143.64 tons/hour
Solid cake: 22.96 tons/hour with 36% of solid material. The solid part is 54.1% of the non-hydrolyzed cellulose.

The ethanol yield of this process is therefore 15.8% by weight on the native vegetation (dry base material). The specific enzyme consumption is 51,540 FPu/kg of ethanol that is produced.

The cellulose called “recalcitrant” that is present in the cake has a hydrolysis yield (under the conditions of the process above) and with the same enzymatic feedstock that will be only 30%.

### Example 2

### Material Balance—with Recycling of the Solid Residue at the Level of the Enzymatic Hydrolysis (Not in Accordance with the Invention)

It is possible to recycle the cake at the enzymatic hydrolysis stage so as to limit the losses of cellulose. Nevertheless, in the absence of treatment, the cellulose called “recalcitrant” has a very reduced sensitivity to enzymatic hydrolysis relative to the cellulose of the papermaking paste. Its hydrolysis yield (under the conditions of the process above), and with the same enzymatic feedstock, will be only 30%. In addition, the recycling brings about the accumulation of insoluble products (lignin) in the process, and the fermentation, under the conditions of the process above, is to be carried out with a maximum of 8% of solid material in the reaction medium. Thus, it is necessary to limit the quantity of recycled cake, and, in practice, only 68% of the cake can be recycled. The following exiting flows of the process are then obtained:
Ethanol at 99.7% by weight: 7.92 tons/hour, or 14% more than Example 1.
Vinasse: 198.06 tons/hour
Solid cake: 17.14 tons/hour with 36% solid material (non-recycled part). The solid part is 50.3% of the non-hydrolyzed cellulose.

The ethanol yield of this process is therefore 18.0% by weight on the native vegetation (dry base material) or an improvement of 2.2 points relative to the basic case. Nevertheless, this improvement of the mass balance is achieved to the detriment of the specific enzyme consumption that is then 61,740 FPu/kg of ethanol produced (+20%) and requires a larger reaction volume: +56% for enzymatic hydrolysis or an increase of the specific volume (relative to the production) of 36%.

Thus, the improvement of the mass balance makes it possible to reduce the contribution of the cost of the raw material in the final production cost of ethanol, but the expense items “enzymes” and “investments” are increased significantly.

### Example 3

### Material Balance—with Recycling within a Regeneration Reactor (According to the Invention)

On the basis of the process that is described in Example 1, a recycling of 90% of the cake that is created is introduced into a regeneration reactor, where the cake undergoes a “mild” baking at 110° C. for 1 hour in the presence of sodium sulfate, before being mixed with the pretreated native vegetation downstream from the pretreatment reactor 2.

Thus, 27.18 tons/hour of moist cake that contains 52.2% of cellulose is recycled at the regeneration reactor. The cost of this type of baking is less than that of the rigorous treatment carried out in the reactor 2 for the pretreatment of native vegetation. Furthermore, the recycled cellulose is no longer much protected by the lignin, since the ligneous sheath that protects the fibers has been largely removed during the pretreatment, and therefore a milder and shorter baking is adequate for imparting to cellulose fibers their full susceptibility to enzymatic hydrolysis, and makes it possible to greatly limit the losses of material. During the stages of dedicated baking and washing, the following are preserved:

| | |
|---|---|
| Cellulose | 95% |
| Lignin | 40% |
| Hemicellulose | 60% |
| Others (Ashes, | 10% |
| Extractibles, . . . ) | |

The hydrolysis and fermentation conditions are preserved. Because of the significant swelling of the cellulose fibers that are recycled in the alkaline medium and in the absence of lignin surrounding these fibers upon their input into the regeneration reactor, the cellulose recovers all of its susceptibility to enzymatic hydrolysis and therefore has a hydrolysis yield that is equal to that of the cellulose that is obtained from the native vegetation (75%). The hemicelluloses also recover a yield of 55%. The conditions of alcohol fermentation and separation are preserved. Thus, owing to the process according to the invention, exiting flows of the process are obtained:
Ethanol at 99.7% by weight: 8.79 tons/hour, or 26% more than Example 1.
Vinasse: 177.10 tons/hour
Solid cake: 3.02 tons/hour with 36% solid material (non-recycled part). The solid part is 52.2% of the non-hydrolyzed cellulose.

The ethanol yield of this process is therefore 20.0% by weight on the native vegetation (dry base material) or 4.2 points more than Example 1 and 2 points more than Example 2. Furthermore, the specific enzyme consumption has only very slightly increased and is 51,750 FPu/kg of ethanol that is produced, or an only 0.4% increase. The reaction volume that is involved is 29% greater than Example 1, and therefore the specific volume has only slightly increased relative to Example 1 (+1.7%).

The implementation of the process according to the invention has made it possible to greatly improve the mass balance and therefore to decrease the contribution of the cost of the raw material in the final production cost of ethanol. The recycling according to the invention makes it possible to monitor the lignin level in the process and therefore makes it possible to recycle a larger quantity than Example 2, while keeping a correct level of solids in fermentation, which leads to an even better material yield. In addition, the invention makes it possible to preserve the contribution of expense items “enzymes” and “investments” in the case without recycling (less than 2% increase). The “mild” baking conditions make it possible to preserve a very large portion of the cellulose while imparting to it its susceptibility to hydrolysis by enzymes, and the cost associated with this baking is lower than that of the pretreatment of the native vegetation.

### Example 4

### Material Balance—without Recycling (Not in Accordance with the Invention)

A process for the production of an acetone-butanol-ethanol (ABE) mixture from papermaking pulp obtained from a Kraft alkaline process is considered. The process treats 150 tons/hour of native vegetation. The vegetation is eucalyptus (hardwood), containing 50% by weight of dry material that consists of:

| | |
|---|---|
| Cellulose | 45% |
| Lignin | 22% |
| Hemicellulose | 17% |
| Others (Ashes, | 16% |
| Extractibles . . . ) | |

The hemicelluloses consist of C5 sugars (xylans and arabinans).

The Kraft baking is carried out at 165° C. for 2.5 hours. This pretreatment and the washing processes carried out in stages a) and b) respectively are conducted in such a way that the papermaking pulp contains 10% dry material, and has preserved the following:

| | |
|---|---|
| Cellulose | 98.5% |
| Lignin | 9% |
| Hemicellulose | 65% |
| Others | 20% |

The ethanol conversion process consists of enzymatic hydrolysis of the papermaking pulp (stage c), a separation of solids in suspension for forming a cake with a washing for maximizing the recovery of sugars, and then an ABE fermentation of the liquid phase that contains the sugars (stage d)), and the distillation of the ABE (stage e). It should be noted that the ABE fermentation uses the sugars both with 6 atoms and with 5 atoms of carbon (glucose and xylose).

The enzymatic hydrolysis is conducted under conditions such that the hydrolysis of 85% of the cellulose and 65% of the hemicelluloses is observed. 25 FPu/g of cellulose entering the hydrolysis reactor is consumed.

Before the fermentation stage, the solid residue is separated and washed for limiting the loss of sugar with the cake.

The fermentation makes it possible to transform the glucose and the xylose previously formed into an ABE mixture, producing 0.3 g of ABE per g of sugar present.

The conditions of the process are such that the exiting flows are:
ABE (pure): 11.16 tons/hour
Vinasse: 416.95 tons/hour
Solid cake: 33.74 tons/hour with 33.3% solid material. The solid part is 44.3% of the non-hydrolyzed cellulose.

The ABE yield of this process is therefore 14.9% by weight on the native vegetation (base ms). The specific enzyme consumption is 74,470 FPu/kg of ABE that is produced.

The cellulose called “recalcitrant” that is present in the cake has a hydrolysis yield (under the conditions of the process above) and with the same enzymatic feedstock that will be only 25%.

### Example 5

### Material Balance—with Recycling of the Solid Residue at the Level of Enzymatic Hydrolysis (Not in Accordance with the Invention)

It is possible to recycle the cake at the enzymatic hydrolysis stage so as to limit the losses of cellulose. Nevertheless, in the absence of treatment, the cellulose called “recalcitrant” has a very reduced sensitivity to the enzymatic hydrolysis relative to the cellulose of the papermaking paste. Its hydrolysis yield (under the conditions of the process above), and with the same enzymatic feedstock, will be only 25%. In addition, the recycling brings about the accumulation of insoluble products (lignin) in the process, which leads to larger volumes of hydrolysis reactors. 90% of the thus formed cake is recycled. The following exiting flows of the process are then obtained:
ABE (pure): 12.84 tons/hour, or 15% more than Example 4.
Vinasse: 787.41 tons/hour
Solid cake: 17.10 tons/hour with 33.3% solid material (non-recycled part). The solid part is 26.9% of the non-hydrolyzed cellulose.

The ABE yield of this process is therefore 17.1% by weight on the native vegetation (dry base material) or an improvement of 2.2 points relative to the basic case. Nevertheless, this improvement of the mass balance is achieved to the detriment of the specific enzyme consumption that is then 91,590 FPu/kg of ABE produced (+23%) and requires a reaction volume that has more than doubled: +113% for enzymatic hydrolysis or an increase of the specific volume (relative to the production) by +85%.

Thus, the improvement of the mass balance makes it possible to reduce the contribution of the cost of the raw material in the final production cost of ABE, but the expense items “enzymes” and primarily “investments” are increased significantly.

### Example 6

### Material Balance—with Recycling of the Solid Residue within a Regeneration Reactor (According to the Invention)

On the basis of the process that is described in Example 4, a recycling of 90% of the cake that is created is introduced into a regeneration reactor, where the cake undergoes a “mild” baking at 105° C. for 45 minutes, in the presence of sodium sulfate, before being mixed with the pretreated native vegetation at the outlet of the pretreatment reactor 2.

Thus, 38.52 tons/hour of moist cake that contains 40.2% of cellulose is recycled at the regeneration reactor. The cost of this type of baking is less than that of the rigorous treatment carried out in the reactor 2 for the pretreatment of native vegetation. Furthermore, the recycled cellulose is no longer much protected by the lignin, since the ligneous sheath that protects the fibers has been largely removed during the pretreatment, and therefore a milder and shorter baking is adequate for imparting to cellulose fibers their full susceptibility to enzymatic hydrolysis, and makes it possible to greatly limit the losses of material. During the stages of dedicated baking and washing, the following are preserved:

| | |
|---|---|
| Cellulose | 97% |
| Lignin | 50% |
| Hemicellulose | 65% |
| Others | 18% |

The hydrolysis and fermentation conditions are preserved. The hydrolysis of the native vegetation has the same yield. Because of the significant swelling of the cellulose fibers that are recycled in the alkaline medium—and in the absence of lignin surrounding these fibers upon their input into the digester for the chemical alkaline pretreatment—the cellulose recovers all of its susceptibility to enzymatic hydrolysis and therefore has a hydrolysis yield that is equal to that of the cellulose that is obtained from the native vegetation (85%). The hemicelluloses also recover a yield of 65%. The conditions of ABE fermentation and separation are preserved. Thus, owing to the process according to the invention, exiting flows of the process are obtained:
ABE (pure): 12.98 tons/hour, or 16.3% more than Example 4.
Vinasse: 489.05 tons/hour
Solid cake: 4.28 tons/hour (non-recycled part), containing 33.3% solids. The solid part is 40.2% of the cellulose.

The ABE yield of this process is therefore 17.3% by weight on the native vegetation (dry base material) or 2.4 points more than Example 4 and 0.2 point more than Example 5. Furthermore, the specific enzyme consumption has only very slightly decreased and is 73,690 FPu/kg of ABE that is produced, or 1% reduction. The reaction volume that is involved is 19.1% greater than Example 4, and therefore the specific volume is slightly greater than for Example 4 (+2.4%).

The implementation of the process according to the invention has made it possible to greatly improve the mass balance and therefore to decrease the contribution of the cost of the raw material in the final production cost of ABE. The recycling according to the invention makes it possible to monitor the lignin level in the process and therefore makes it possible to limit the volume that is necessary to the hydrolysis relative to Example 5. In addition, the invention makes it possible to preserve the contribution of expense items “enzymes” and “investments” in the case without recycling (less than 3% difference). The “mild” baking conditions make it possible to preserve a very large portion of the cellulose while imparting to it its susceptibility to hydrolysis by enzymes, and the cost associated with this baking is lower than that of the pretreatment of the native vegetation.

1-16. (canceled)

17. A process for the production of alcohols and/or solvents from cellulosic or lignocellulosic biomass that comprises at least the following stages:
a) Alkaline chemical pretreatment of a cellulosic or lignocellulosic substrate;
b) Optionally washing of the pretreated substrate;
c) Enzymatic hydrolysis of the substrate that is pretreated and optionally washed using cellulolytic and/or hemicellulolytic enzymes that produce a hydrolyzate and a water-insoluble residue;
d) Microorganism fermentation of the hydrolyzate that is obtained from stage c) and production of a fermentation must that contains at least one alcohol and/or solvent;
e) Separation/purification of alcohol and/or solvent, and
f) Separation of a cake that contains insoluble residue,
in which at least a portion of the cake that is obtained in stage f) is sent into at least one reactor for regeneration of cellulose, before being recycled downstream from stage a) for alkaline chemical pretreatment where it is mixed with alkaline solution and heated to 50-150° C. and
wherein regeneration is carried out without a stream obtained from the pre-treatment of the feed.

18. Production process according to claim 17, in which at least one fraction that represents between 20 and 100% of the cake is sent into said regeneration reactor.

19. Process according to claim 18, in which the at least one fraction represents between 75 and 100% of the cake.

20. The process according to claim 17, in which at least one fraction that represents between 0 and 80% of the cake is directly discharged without recycling.

21. The process according to claim 20, in which the at least one fraction represents less than 25% of the flow of insoluble residue.

22. The process according to claim 17, in which the a fraction is mixed with an alkaline solution in the regeneration reactor and then heated to a temperature of between 70 and 150° C. for a period that varies between 10 minutes and 4 hours.

23. The process according to claim 22, in which said alkaline solution is sodium sulfate, with the temperature in said reactor being between 70 and 150° C., and the dwell time of between 0.5 and 4 hours.

24. The process according to claim 22, in which said alkaline solution is gaseous ammonia, with the temperature being between 50 and 100° C., and the dwell time being between 10 and 60 minutes.

25. The process according to claim 22, in which said alkaline solution is a percolated ammoniacal solution, heated to a temperature of between 80 and 140° C.

26. The process according to claim 17, in which the pretreatment that is carried out in stage a) is a pretreatment with sodium sulfate, also called a Kraft process, a pretreatment by explosion of the fibers with ammonia, also called AFEX pretreatment, or a percolation pretreatment that uses ammonia with recycling, also called ARP pretreatment.

27. The process according to claim 17, in which the washing stage b) is necessary if the alkaline pretreatment is a Kraft- or ARP-type pretreatment.

28. The process according to claim 17, in which stage c) for enzymatic hydrolysis is carried out by means of cellulases and/or hemicellulases that are produced by a microorganism that is a fungus that belongs to the genera Trichoderma, Aspergillus, Penicillium or Schizophyllum, or an anaerobic bacteria that belongs to the genus Clostridium.

29. The process according to claim 17, in which stage c) is carried out in such a way that between 20 and 90%, of the cellulose that is contained in the pretreated and optionally washed substrate is converted into glucose.

30. The process according to claim 17, in which the alcohol that is obtained at the end of stage e) is ethanol.

31. The process according to claim 17, in which the solvent that is obtained at the end of stage e) is an acetone-butanol-ethanol mixture.

32. The process according to claim 18, wherein stage f) separation of the cake is carried out downstream from stages c), d) and/or e) and optionally is coupled to a washing of the cake.

33. The process according to claim 17, wherein the temperature during regeneration is lower than the temperature during pre-treatment.

34. The process according to claim 17, wherein the temperature during regeneration is from 50° C. to 150° C. and during pre-treatment is from 150° C. to 180° C.
